(19) United States
(12) Patent Application Publication
(10) Pub. No.: US 2018/0085555 A1
(43) Pub. Date: Mar. 29, 2018

(54) INJECTION CATHETER

(71) Applicant: BOSTON SCIENTIFIC SCIMED, INC., MAPLE GROVE, MN (US)

(72) Inventors: AIDEN FLANAGAN, CO. GALWAY (IE); CONOR GREANEY, CO. GALWAY (IE); BRUCE PHILIP MURPHY, CO. DUBLIN (IE); EIMEAR BREEGE DOLAN, CO. MAYO (IE)

(73) Assignee: BOSTON SCIENTIFIC SCIMED, INC., MAPLE GROVE, MN (US)

(21) Appl. No.: 15/715,054

(22) Filed: Sep. 25, 2017

Related U.S. Application Data
(60) Provisional application No. 62/399,696, filed on Sep. 26, 2016.

(51) Int. Cl. A61M 25/00; A61M 25/01; A61M 25/06

(52) U.S. Cl. CPC A61M 25/0071; A61M 25/0147; A61M 2210/125; A61M 25/0662; A61M 25/0084

(57) ABSTRACT

An example medical device and method of use is disclosed. An example medical device includes a catheter having a distal end region, a proximal end region and a lumen extending therein. The medical device further includes a tip member disposed along the distal end region, the tip member extending radially inward from a wall surface of the catheter. The medical device further includes an inner member slidably disposed within the lumen of the catheter, the inner member having a first lumen, a second lumen, a mixing region, and a distally extending member, wherein an opening is defined in a central region of the tip member, and wherein the distally extending member is designed to extend through the opening.

### CROSS-REFERENCE TO RELATED APPLICATIONS

This application claims the benefit of priority under 35 U.S.C. § 119 to U.S. Provisional Application Ser. No. 62/399,696, filed Sep. 26, 2016, the entirety of which is incorporated herein by reference.

### TECHNICAL FIELD

This disclosure pertains to delivering a therapeutic treatment to a target area in a patient's body such as cardiac tissue.

### BACKGROUND

Heart failure due to damaged cardiac tissue is a significant health care issue. It has been proposed to treat the damaged tissue directly with a therapeutic agent designed to help regenerate the damaged tissue. An example of a therapeutic agent proposed for this use is stem cells. The stem cells may be delivered in the form of a gel to the site of the damaged tissue. The gels may be administered via an injection catheter having an extendable tip designed to precisely position the cells adjacent to the target site.

### SUMMARY

This disclosure provides design, material, manufacturing method, and use alternatives for medical devices. An example medical device includes a catheter having a distal end region, a proximal end region and a lumen extending therein. The medical device further includes a tip member disposed along the distal end region, the tip member extending radially inward from a wall surface of the catheter. The medical device further includes an inner member slidably disposed within the lumen of the catheter, the inner member having a first lumen, a second lumen, a mixing region, and a distally extending member, wherein an opening is defined in a central region of the tip member, and wherein the distally extending member is designed to extend through the opening.

Alternatively or additionally to any of the embodiments above, further comprising a pull wire disposed along the inner member.

Alternatively or additionally to any of the embodiments above, wherein the inner member further includes a wall portion, and wherein the pull wire is disposed within the wall portion.

Alternatively or additionally to any of the embodiments above, wherein the inner member includes a distally facing shoulder, wherein the tip member includes a proximally facing shoulder, and wherein the proximally facing shoulder is configured to mate with the distally facing shoulder.

Alternatively or additionally to any of the embodiments above, wherein a length is defined between the distally facing shoulder and the proximally facing shoulder, and wherein the length defines a maximum distance that the distally extending member extends into a target site.

Alternatively or additionally to any of the embodiments above, further comprising a spring disposed between the distally facing shoulder and the proximally facing shoulder, wherein the spring is configured to bias the distally extending member in a retracted position.

Alternatively or additionally to any of the embodiments above, wherein the distally extending member includes a needle.

Alternatively or additionally to any of the embodiments above, further comprising a second pull wire, and wherein the second pull wire is disposed along the catheter. Alternatively or additionally to any of the embodiments above, wherein the inner member is configured to rotate independently of the catheter.

Alternatively or additionally to any of the embodiments above, wherein the mixing region is positioned distally of the first lumen and the second lumen. Alternatively or additionally to any of the embodiments above, wherein the inner member further comprises a tip portion, wherein the tip portion is configured to separate from the inner member.

Alternatively or additionally to any of the embodiments above, wherein the mixing region is located within the tip portion.

Another example medical device includes:

a guide sheath having a distal end region and a lumen extending therein;

a shoulder disposed along the distal end region, wherein the shoulder extends radially inward from an inner wall of the guide sheath;

a mixing tube having a distal portion and a plurality of lumens extending therein, wherein the mixing tube includes a distally facing surface configured to mate with the shoulder; and

a needle extending distally from the distally facing surface;

wherein the shoulder defines an aperture;

wherein the needle is configured to extend through the aperture.

Alternatively or additionally to any of the embodiments above, further comprising a pull wire disposed along the mixing tube.

Alternatively or additionally to any of the embodiments above, further comprising a second pull wire, and wherein the second pull wire is disposed along the guide sheath. Alternatively or additionally to any of the embodiments above, wherein the mixing tube is configured to rotate independently of the guide sheath.

Alternatively or additionally to any of the embodiments above, further comprising a mixing region positioned distally of the first lumen and the second lumen.

Alternatively or additionally to any of the embodiments above, wherein the mixing tube further comprises a tip portion, wherein the tip portion is configured to separate from the mixing tube.

Alternatively or additionally to any of the embodiments above, further comprising a spring disposed between the shoulder and the distally facing surface, wherein the spring is configured to bias the needle in a retracted position.

Another example medical device includes:

a catheter having a distal end region, a proximal end region and a lumen extending therein;

a tip member disposed along the distal end region, the tip member extending radially inward from a wall surface of the catheter; and

an inner member slidably disposed within the lumen of the catheter, the inner member having a first treatment disposed in a first lumen, a second treatment disposed in a second lumen, a mixing region configured to combine the first treatment and the second treatment, and a distally extending member;

wherein an opening is defined in a central region of the tip member;

wherein the distally extending member is designed to extend through the opening.

Alternatively or additionally to any of the embodiments above, further comprising a pull wire disposed along the inner member.

The above summary of some embodiments is not intended to describe each disclosed embodiment or every implementation of the present disclosure. The Figures, and Detailed Description, which follow, more particularly exemplify these embodiments.

### DESCRIPTION OF DRAWINGS

FIG. 1 illustrates an example injection catheter positioned adjacent a treatment site in a heart.

FIG. 2 illustrates an example injection catheter.

FIG. 3 illustrates a cross-sectional view along line 3-3 in FIG. 1.

FIG. 4 illustrates another example injection catheter.

FIG. 5 illustrates another example injection catheter.

FIG. 6 illustrates another example injection catheter.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Congestive heart failure—the ineffective pumping of the heart caused by the loss or dysfunction of heart muscle cells—afflicts millions of people each year. One of the major contributors to the development of congestive heart failure is myocardial infarction. Myocardial infarction destroys heart muscle cells, known as cardiomyocytes, and can be the result of hypertension, chronic insufficiency in the blood supply to the heart muscle caused by coronary artery disease, or a heart attack, the sudden closing of a blood vessel supplying oxygen to the heart. In some instances, it may be beneficial to repair and/or restore diseased heart tissue in an effort to improve the pumping efficiency of the heart muscle. For example, in some instances stem cells may be utilized to replace dead or impaired cells so that the weakened heart muscle can regain its full pumping power. The injection catheters disclosed herein may deliver therapeutics, such as gels including tissues, biologics, stem cells, poietic cells, or fully diversified cells, to a treatment location, such as a heart wall or other organ. For example, cardiac cells could be injected into the heart tissue or liver cells injected into the liver.

In some cases, the therapeutics delivered using examples provided herein may include mixing different gel components to create a gel or solid. These gels and solids may result in the clogging of passages in an injection device or system. Therefore, it may be desirable to maintain gels in separate catheter lumens prior to mixing and injection into the heart tissue. Further, in some cases, the examples disclosed herein may include a detachable tip that may be replaced in the event that two or more gels solidify and cannot be injected via the tip. For example, in some cases a detachable tip may be separated from the remainder of a device provided herein between injections to clean it out. In other cases, a detachable tip may be separated from a remainder of a device provided herein between injections to replace it with a new detachable tip

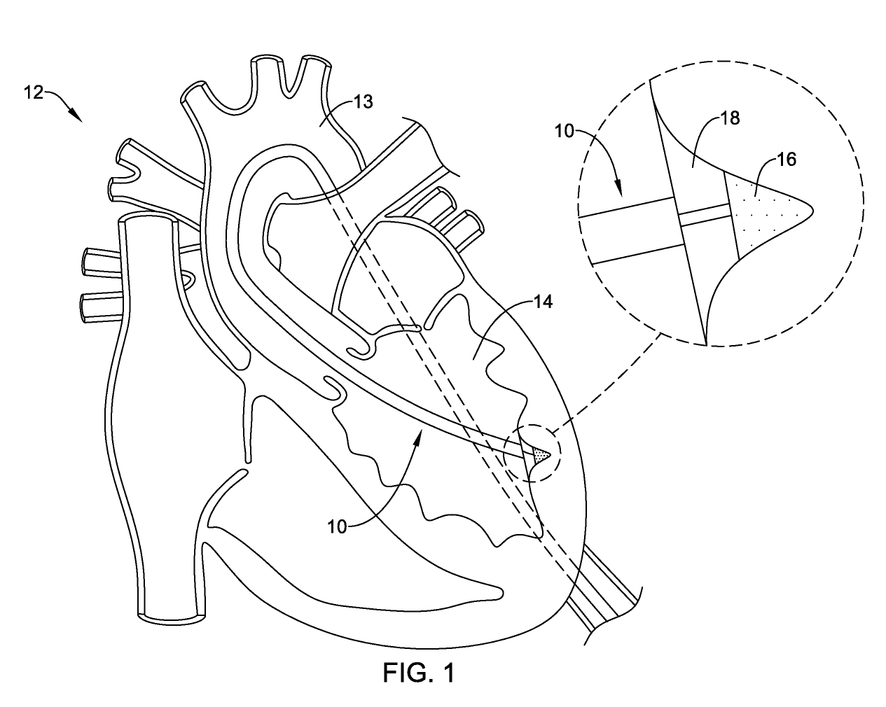

FIG. 1 illustrates an example injection catheter system 10 utilized to deliver a treatment (e.g., therapeutic agents) to a left ventricle wall 14 of a heart 12. The catheter system 10 may be positioned adjacent a target site by advancing injection catheter system 10 through the aorta 12. The detailed view in FIG. 1 illustrates that the distal portion of the catheter system 10 may be positioned near a cardiac treatment site (heart wall tissue). For example, the distal end of the catheter system 10 may be positioned adjacent (e.g., abutted against) diseased cardiac tissue which may include pockets (e.g., cavities) 18 in which the treatment 16 may be injected.

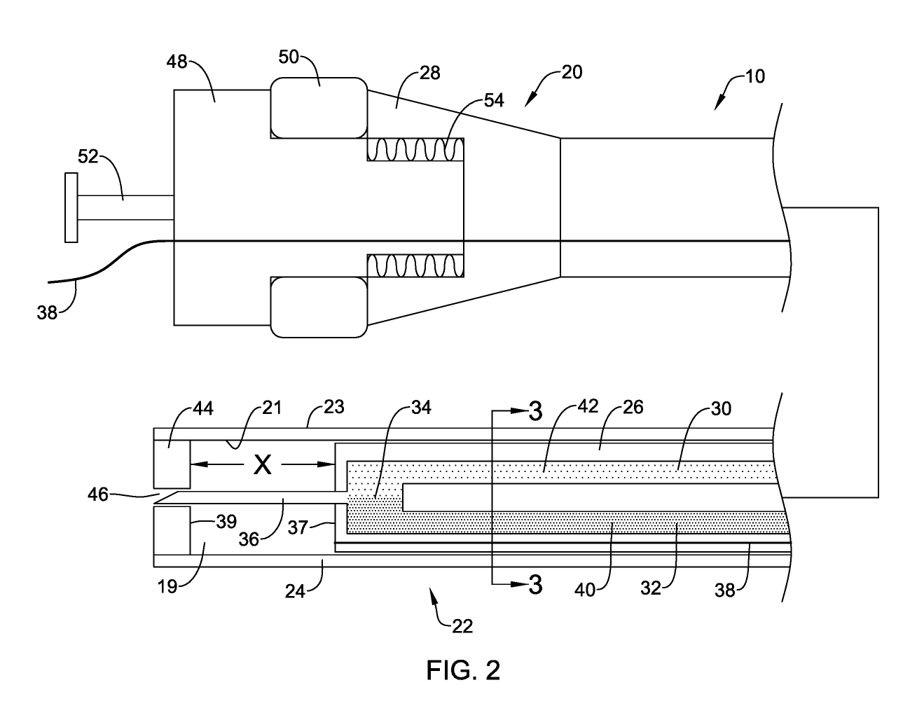

FIG. 2 illustrates the example catheter system 10 shown in FIG. 1. In some examples, the example catheter system 10 illustrated and described herein may be referred to as an injection catheter 10 or injection catheter system 10. The injection catheter 10 may include a proximal region 20 and a distal region 22. The injection catheter 10 shown in FIG. 2 may further include an outer member 24. Outer member 24 may be referred to as a guide sheath, guide catheter and/or outer tubular member 24. Injection catheter system 10 may also include a proximal member 28. In some instances, proximal member 28 may be described as a manifold 28 or handle member 28. As shown in FIG. 2, a proximal portion of outer member 24 may be coupled to a distal portion of proximal member 28.

Outer member 24 may further include a lumen 19 extending from the proximal region 20 to the distal region 22 of injection catheter 10. For example, lumen 19 may extend along the entire length of outer member 24 or may only extend along a portion of outer member 24. Further, as illustrated in FIG. 2, outer member 24 may include a wall thickness extending between an inner surface 21 and an outer surface 23 of outer member 24.

FIG. 2 further illustrates an inner member 26 slideably disposed within lumen 19 of outer member 24. Inner member 26 may be able to translate along the longitudinal axis of injection catheter 10. In addition, inner member 26 may be able to rotate within lumen 19 of outer member 24. As shown in FIG. 2, inner member 26 may include a first lumen 30 and a second lumen 32. While illustrated with two lumens, it is contemplated that inner member 26 may include more or less than two lumens. For example, inner member 26 may include 1, 2, 3, 4, 5 or more lumens.

Inner member 26 may extend with lumen 19 along the entire length of outer member 24. For example, inner member 26 may be slidably disposed within outer member 24 from the distal region 22 to the proximal region 20. Additionally, inner member 26 may be coupled to an injection handle 48. For example, the proximal portion of inner member 26 may be coupled to the distal end of injection handle 48. Injection handle 48 may be used to translate inner member 26 along the longitudinal axis of injection catheter system 10. For example, distal advancement of injection handle 48 may advance inner member 26 in a distal direction. Further, retraction of injection handle 48 may retract inner member 26 in a proximal direction. It can be appreciated that injection handle 48 may translate along the longitudinal axis of catheter 10 relative to proximal member 28. Additionally, it is contemplated that that injection handle 48 may include additional features that facilitate relative motion with proximal member 28. For example, injection handle 48 may include threads (not shown in FIG. 2) that mate with opposing threads (not shown in FIG. 2) on proximal member 28. The threaded connection may permit fine motion control of injection handle 48 relative to proximal member 28.

As illustrated in FIG. 2, inner member 26 may further include a mixing region 34. In some examples, mixing region 34 may be located distally of lumen 30 and lumen 32. In other words, mixing region 34 may be defined as a portion of the lumen of inner member in which a first treatment (e.g., first gel) 40 may be mixed (e.g., combined) with a second treatment (e.g., second gel) 42. It can be appreciated that as first and second treatments 40/42 are advanced distally through first and second lumens 30/32, they may combine within mixing region 34. Additionally, it is contemplated that first and second treatments 40/42 may be partially mixed in mixing region 34. After partial mixing in mixing region 34, treatments 40/42 may be fully mixed within a distally extending member 36 (described below).

FIG. 2 further illustrates a plunger member 52 extending proximally from the proximal region 20 of injection catheter 10. For example, FIG. 2 shows a plunger member 52 extending proximally from injection handle 48. It can be appreciated that plunger member 52 may be used to advance first treatment 40 and second treatment 42 along lumens 30/32. In other words, plunger member 52 may be translated proximally along the longitudinal axis of injection catheter system 10. The distal translation (e.g., pushing plunger member 52 in a distal direction) may advance first treatment 40 along lumen 32 and second treatment 42 along lumen 30. The distal advancement of the first treatment 40 and second treatment 42 may push the first and second treatments 40/42 into the mixing region 34, whereby the first and second treatments 40/42 may combine with one another to form a mixed treatment which may be injected into a treatment site (e.g., cardiac tissue).

FIG. 2 illustrates a distally extending member 36 disposed along a distal end of inner member 26. In some examples disclosed herein, distally extending member 36 may include a needle. As shown in FIG. 2, distally extending member 36 may be positioned in a central region of the distal end of inner member 26. Distally extending member 36 may be in fluid communication with mixing region 34. In other words, a treatment (e.g., gel), may be advanced distally away from mixing region 34, through distally extending member 36 and into a target site (e.g., injected into heart tissue).

FIG. 2 further illustrates that outer member 24 may include a tip member 44 extending radially inward from the inner surface 21 of outer member 24. Tip member 44 may include an aperture 46 positioned in a central region of tip member 44. Further, FIG. 2 illustrates that, in at least some examples disclosed herein, distally extending member 36 may be aligned with the aperture 46 in tip member 44. For example, FIG. 2 shows needle 36 disposed within aperture 46 of tip member 44.

In some instances, it may be desirable to bias the inner member 26 of injection catheter 10 such that inner member 26 (including distally extending tip 36) is disposed within outer member 24. In other words, it may be desirable to bias inner member 26 in a proximal-most direction within outer member 24. While various design configurations are contemplated to achieve such bias, FIG. 2 shows a proximal spring member 54 disposed between injection member 48 (which is coupled to inner member 26) and proximal member 28. It can be appreciated that proximal spring member 54 may be biased to expand outwardly, thereby exerting a force upon proximal member 28 that pushes injection member 48 in a proximal direction. Because injection member 48 is coupled to inner member 26, a proximally-directed force placed upon injection member 48 will also act upon inner member 26.

It can be appreciated that spring 54 may be tailored such that an operator or clinician will be able to overcome the force that spring 54 exerts on injection member 48. For example, in order to advance extending member 36 distally (e.g., through aperture 46), an operator or clinician may squeeze (e.g., press) injection member 48 and proximal member 28 toward one another. As shown in FIG. 2, in some instances a portion of injection member 48 may project into and mate with a portion of proximal handle 28. If the operator exerts a force strong enough to overcome the resistance of spring 54, injection member 48 may advance distally, thereby also advancing needle 36 distally through aperture 46.

Additionally, FIG. 2 shows a safety lock 50 disposed on injection member 48. Safety lock 50 may be configured to be placed around a portion of the circumference of injection member 48. Safety lock 50 may be releasably coupled to injection member 48. It can be appreciated that when safety lock 50 is coupled to injection member 48, it may prevent the inadvertent advancement of injection member 48 in a distal direction (e.g., toward proximal member 28). In other words, safety lock 48 may be present on injection catheter system 10 prior to being used in a surgical procedure, and therefore, removal of the safety lock 50 (e.g., by an operator or clinician) may need to be performed prior to performing an injection into a target site. In some instances, it may be desirable to be able to steer and/or direct the distal portion of injection catheter 10 to a target site. Therefore, in at least some examples disclosed herein, a pull wire 38 may disposed along a portion of inner member 26. For example, FIG. 2 shows a pull wire 38 disposed within the wall of inner member 26. This is not intended to be limiting. Rather, it is contemplated that one or more pull wires 38 may be disposed along any portion of catheter system 10, including inner member 26. For example, pull wire 38 may be disposed along the inner surface and/or outer surface of inner member 26. Additionally, it is contemplated that one or more pull wires may be disposed along other members of catheter system 10. For example, while not shown in the Figures, it is contemplated that one or more pull wires 38 may be disposed along outer member 24. Further, one or more pull wires 38 may be located only in the inner member 26, only in the outer member 24 or in both the inner and outer members 26/24.

Pull wire 38 may extend along inner member 26 to a portion proximal of injection member 48. In other words, when a clinician is using injection catheter 10 during a procedure, proximal member 28, injection member 48, plunger 52 and pull wire 38 may be positioned outside the patient's body. A clinician may manipulate a proximal portion of pull wire 38 which may, in turn, steer and/or direct a distal portion of inner member 26. For example, as a clinician retracts and/or rotates pull wire 38, the distal end of inner member 26 may move laterally and/or rotate.

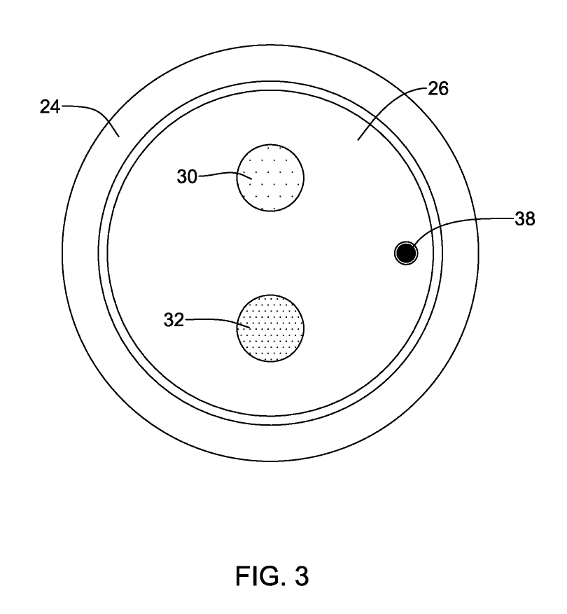

FIG. 3 illustrates a cross-section along line 3-3 of FIG. 2. FIG. 3 illustrates first lumen 30 and second lumen 32 of inner member 26 as described above. Further, FIG. 3 shows pull wire 38 extending with the wall portion of inner member 26 as described above. Further, FIG. 3 shows inner member 26 disposed within the lumen of outer member 24.

Aperture 46 may be configured such that distally extending member 36 may track through aperture 46 as injection handle 48 is actuated to extend needle 36 in a distal direction. Referring back to FIG. 2, it can be appreciated that tip member 44 may include a proximally facing surface 39 and inner member 26 may include a shoulder 37 (e.g., a distally facing surface). Further, FIG. 2 illustrates a distance depicted as “X” between the proximal facing surface 39 and the shoulder 37. It can be appreciated that the proximal facing surface 39 may mate with the shoulder 37. In other words, as shoulder 37 is advanced in a distal direction, it may stop when it contacts proximal facing surface 39. Further, it can be appreciated that the maximum distance in which inner member 26 may travel distally within the lumen 19 of outer member 24 will be equal to distance “X.”

It should be noted that in some examples it may be beneficial to design system 10 such that distance “X” is less that the length (as measured along the longitudinal axis of device 10) of safety lock 50. For example, it may be desirable to design system 10 such that the distal tip of the distally extending member 36 remain proximal of the distal face of tip member 44. Further, when positioned in a substantially linear configuration, it may be desirable that the distal tip of distally extending member 36 be positioned proximally inward of the distal face of tip member 44 (e.g, proximally inward 1 mm-5 mm from the distal face of tip member 44). Positioning the distal tip of distally extending member 36 proximally inward from the distal face of tip member 44 may prevent the distal tip of distally extending member 36 from protruding beyond the distal face of tip member 44 when system 10 is positioned in curved configuration (e.g., bent).

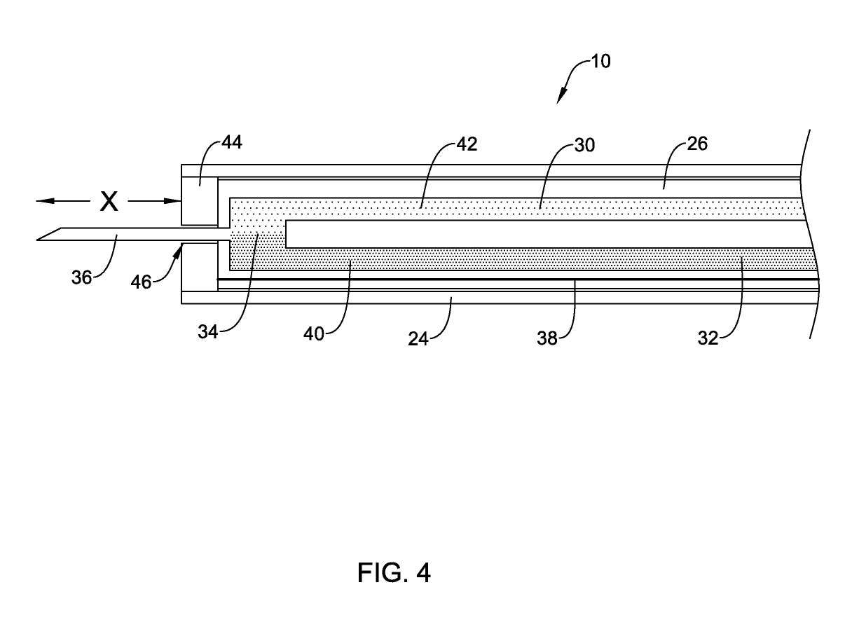

FIG. 4 shows the inner member 26 advanced to a position in which shoulder 37 abuts proximally facing surface 39. Additionally, FIG. 4 shows distally extending member 36 advanced in distal direction through aperture 46. Based on the discussion above regarding the maximum distance “X” that inner member 26 may travel within outer member 24, it can be appreciated the distal end of needle 36 has been advanced distally a corresponding distance “X” past the distal end of outer member 26. This distance “X” is depicted in FIG. 4. This distance may correspond to the maximum distance that needle 36 may be advanced into the tissue of a target site (e.g., tissue of a diseased heart wall).

In some instances it may be difficult for a user to ascertain when the distal face 37 of outer member 24 contacts proximally facing surface 39 of tip member 44. In particular, this issue may arise with longer length injection catheters. Therefore, in some instances it may be desirable to design system 10 to include features that indicate (to a user, for example) when distal face 37 of outer member 24 contacts proximally facing surface 39 of tip member 44. For example, markings on handle member 48 may be utilized to track the distal travel of distal face 37. Additionally, one or more electrical contacts may be provided on both the distal face 37 of outer member 24 and the proximally facing surface 39 of tip member 44. Contact of the electrical contacts with one another may complete an electrical circuit which relays an electrical signal to a user indicating that distal face 37 of outer member 24 has contacted the proximally facing surface 39 of tip member 44. Further, a contrast liquid (e.g., radiopaque liquid) may be added to lumen 19, thereby allowing a user to monitor the displacement of inner member 26 via fluoroscopy. It is further contemplated that one or more apertures may be located in the lumen wall of outer member 24 to permit the ejection of radiopaque liquid out of lumen 19 as inner member 26 is moved proximally toward surface 39.

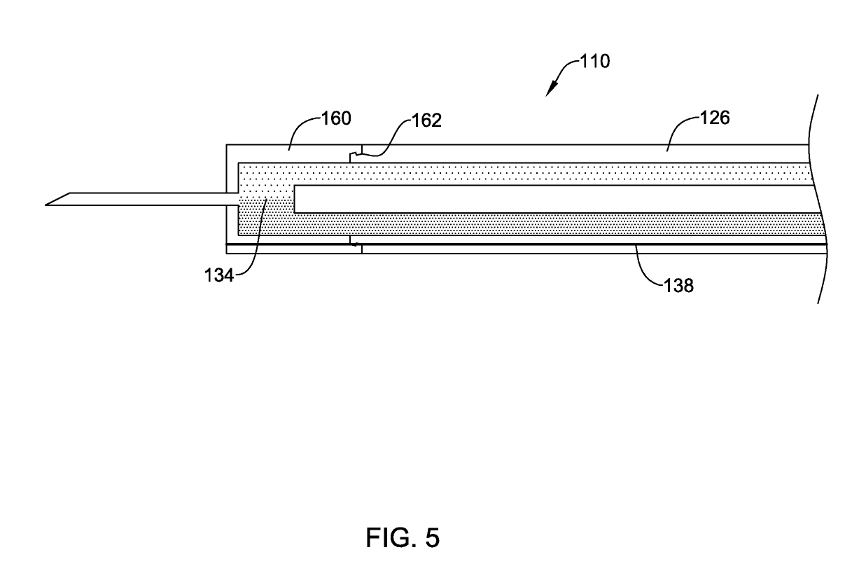

FIG. 5 shows another example injection catheter 110. Injection catheter 110 may be similar in form and functionality to other example injection catheters already described herein. Further, as discussed above, in some instances it may be desirable to design a portion of injection catheter 110 to include a removable tip member 160. For example, FIG. 5 shows example inner member 126 including a removable inner tip member 160. Further, FIG. 5 shows that removable inner tip member 160 may be coupled to a proximal portion of the inner member 126 via a threaded connection 162. It can be appreciated that threaded connection 162 may allow removable inner tip member to be “unscrewed” from the proximal portion of inner member 126.

As discussed above, in some instances a first treatment (e.g., first gel) may be combined with a second treatment (e.g., second gel) to form a combined treatment in the mixing region 134. This mixed treatment may, over time, transform into a highly viscous fluid and/or a solid. Therefore, it may be desirable to position threaded connection 162 at a location proximal to where a clog or obstruction may be located (e.g., the mixing region 134). FIG. 4 shows threaded connection 162 located proximal to mixing region 134. Therefore, if a clog and/or obstruction occurs in mixing region 134 of inner member 126, it is contemplated that the clogged tip member 160 may be replaced by a new, unobstructed, replacement tip member 160.

While the above discussion discloses removing tip member 160 via a threaded connection, it is contemplated that other methods of designing a removable tip member 160 are contemplated. For example, tip member 160 may be attached via a snap connection, press fit connection, or the like.

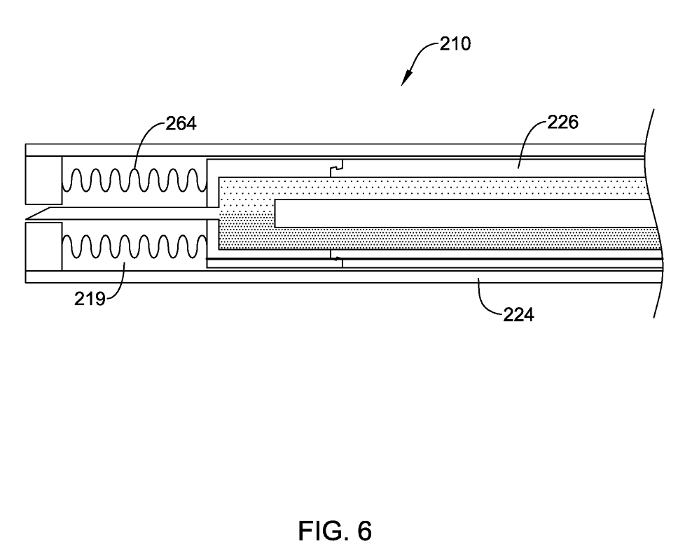

FIG. 6 illustrates another example injection catheter 210. Injection catheter 210 may be similar in form and functionality to other injection catheters and injection catheter systems described herein. For example, injection catheter 210 may include an inner member 226 disposed within a lumen 219 of an outer member 224. Further, injection catheter 210 illustrates a distal spring member 264 positioned in the lumen of outer member 224. As shown in FIG. 6, distal spring member 264 may be positioned between a distal end of inner member 226 and a tip member of outer member 224. In similar fashion to the proximal spring member 54 described with respect to FIG. 2, distal spring member 264 may be biased to expand outwardly, thereby exerting a force upon the proximally facing surface of the tip member of outer member 226 and a proximally-directed force on inner member 226, thereby biasing the inner member 226 in a proximal direction. It can be appreciated that utilizing spring member 264 to bias inner member 226 in a proximal direction allows the distally extending portion to automatically retraction after being inserted into a target site. It can further be appreciated that distal spring member 264 may be tailored such that an operator or clinician will be able to overcome the force that spring 264 exerts on inner member 226. For example, after positioning the distal end of outer member 224 adjacent a target site, an operator may exert a force that overcomes spring 264 (thereby distally advancing inner member 226 and the distally-extending portion into a target site). After being injected into the target site, the needle may automatically retract into the outer member 224 due to the proximal-direction bias provided by spring member 264.

The materials that can be used for the various components of system 10/110 (and/or other systems disclosed herein) and the various tubular members disclosed herein may include those commonly associated with medical devices. For simplicity purposes, the following discussion makes reference to system 10/110 and other components of system 10/110. However, this is not intended to limit the devices and methods described herein, as the discussion may be applied to other similar tubular members and/or components of tubular members or devices disclosed herein.

While the above discussion has focused on examples in which the inner members 26/126/226 are utilized in conjunction with outer members 24/124/224, it is contemplated that any of the inner members or outer members disclosed herein may be utilized individually and/or include any of the components described herein. For example, it is contemplated that inner members 26/126/226 may be advanced to a target independent of an outer member 24/124/224.

System 10/110 and/or other components of system 10/110 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of system 10/110 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of system 10/110 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of system 10/110 to achieve the same result.

In some embodiments, a degree of Magnetic Resonance Imaging (MRI) compatibility is imparted into system 10/110. For example, system 10/110, or portions thereof, may be made of a material that does not substantially distort the image and create substantial artifacts (e.g., gaps in the image). Certain ferromagnetic materials, for example, may not be suitable because they may create artifacts in an MRI image. System 10/110, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
a catheter having a distal end region, a proximal end region and a lumen extending therein;
a tip member disposed along the distal end region, the tip member extending radially inward from a wall surface of the catheter; and
an inner member slidably disposed within the lumen of the catheter, the inner member having a first lumen, a second lumen, a mixing region, and a distally extending member;
wherein an opening is defined in a central region of the tip member;
wherein the distally extending member is designed to extend through the opening.

2. The medical device of claim 1, further comprising a pull wire disposed along the inner member.

3. The medical device of claim 1, wherein the inner member further includes a wall portion, and wherein the pull wire is disposed within the wall portion.

4. The medical device of claim 1, wherein the inner member includes a distally facing shoulder, wherein the tip member includes a proximally facing shoulder, and wherein the proximally facing shoulder is configured to mate with the distally facing shoulder.

5. The medical device of claim 4, wherein a length is defined between the distally facing shoulder and the proximally facing shoulder, and wherein the length defines a maximum distance that the distally extending member extends into a target site.

6. The medical device of claim 5, further comprising a spring disposed between the distally facing shoulder and the proximally facing shoulder, wherein the spring is configured to bias the distally extending member in a retracted position.

7. The medical device of claim 1, wherein the distally extending member includes a needle.

8. The medical device of claim 1, further comprising a second pull wire, and wherein the second pull wire is disposed along the catheter.

9. The medical device of claim 1, wherein the mixing region is positioned distally of the first lumen and the second lumen.

10. The medical device of claim 1, wherein the inner member further comprises a tip portion, wherein the tip portion is configured to separate from the inner member.

11. The medical device of claim 9, wherein the mixing region is located within the tip portion.

12. A medical device, comprising:
a guide sheath having a distal end region and a lumen extending therein;
a shoulder disposed along the distal end region, wherein the shoulder extends radially inward from an inner wall of the guide sheath;
a mixing tube having a distal portion and a plurality of lumens extending therein, wherein the mixing tube includes a distally facing surface configured to mate with the shoulder; and
a needle extending distally from the distally facing surface;
wherein the shoulder defines an aperture;
wherein the needle is configured to extend through the aperture.

13. The medical device of claim 12, further comprising a pull wire disposed along the mixing tube.

14. The medical device of claim 12, further comprising a second pull wire, and wherein the second pull wire is disposed along the guide sheath.

15. The medical device of claim 12, wherein the mixing tube is configured to rotate independently of the guide sheath.

16. The medical device of claim 12, further comprising a mixing region positioned distally of the first lumen and the second lumen.

17. The medical device of claim 12, wherein the mixing tube further comprises a tip portion, wherein the tip portion is configured to separate from the mixing tube.

18. The medical device of claim 12, further comprising a spring disposed between the shoulder and the distally facing surface, wherein the spring is configured to bias the needle in a retracted position.

19. A medical device, comprising:
a catheter having a distal end region, a proximal end region and a lumen extending therein;
a tip member disposed along the distal end region, the tip member extending radially inward from a wall surface of the catheter; and
an inner member slidably disposed within the lumen of the catheter, the inner member having a first treatment disposed in a first lumen, a second treatment disposed in a second lumen, a mixing region configured to combine the first treatment and the second treatment, and a distally extending member;
wherein an opening is defined in a central region of the tip member;
wherein the distally extending member is designed to extend through the opening.

20. The medical device of claim 19, further comprising a pull wire disposed along the inner member.
